(19) United States
(12) Patent Application Publication
(10) Pub. No.: US 2025/0206723 A1
(43) Pub. Date: Jun. 26, 2025

(54) METHODS AND PROCESSES FOR THE PREPARATION OF MCT4 INHIBITORS

(71) Applicant: Vettore, LLC, San Francisco, CA (US)

(72) Inventors: Jon LAWSON, Wildwood, MO (US); Jinxu SUN, Beijing (CN); Guoquan XU, Beijing (CN)

(21) Appl. No.: 19/047,396

(22) Filed: Feb. 6, 2025

Related U.S. Application Data
(63) Continuation of application No. PCT/CN2022/111488, filed on Aug. 10, 2022.

(51) Int. Cl. C07D 403/10; A61K 31/4155

(52) U.S. Cl. CPC C07D 403/10; A61K 31/4155

(57) ABSTRACT

Provided are processes for preparing heterocyclic compounds and compositions for use as MCT4 inhibitors.

This application is a bypass continuation of International Application No. PCT/CN2022/111488, filed Aug. 10, 2022, the disclosure of which is hereby incorporated by reference as if written herein in its entirety.

Disclosed herein are processes for preparing heterocyclic compounds and compositions for use as MCT4 inhibitors.

Lactic acid export from glycolytic cells is typically mediated by the monocarboxylate transporter MCT4. MCT4 exhibits weak affinity for lactate (Kₘ=28 mM) coupled with a high turnover rate, allowing rapid export of large amounts of lactic acid. MCT4 expression is normally limited to highly glycolytic tissues such as white muscle fibers, lymphocytes, astrocytes, and Sertoli cells. Though MCT4 is absent from most normal tissues, MCT4 expression is highly upregulated, and correlates with poor survival, in many cancer indications, including colorectal cancer, glioma, head and neck cancer, triple-negative breast cancer, prostate cancer, KRAS mutant lung cancer, liver cancer, and kidney cancer.

The correlation of MCT4 expression and poor cancer outcome appears to be of significant functional consequence in multiple cancer models. Stable expression of MCT4 is highly tumorigenic in a respiration-impaired, Ras-transformed fibroblast xenograft model. Conversely, MCT4 silencing slows or ablates tumor growth in xenograft models of breast cancer, colorectal cancer, and glioma. MCT4 expression is required for inflammatory cytokine IL-8-mediated angiogenesis in breast and colon cancer xenograft models. MCT4 has also been shown to play important roles in cancer cell migration, invasion, and various aspects of the Warburg effect (e.g., proliferation on glucose, extracellular acidification, and lactate secretion).

Inhibition of MCT4-mediated lactic acid export may be an effective strategy to impair the Warburg effect in cancer. Unfortunately, no potent and selective MCT4 inhibitors have been described. Moderate to weak MCT4 inhibitors are known (e.g., phloretin and α-CN-4-OH-cinnamate); however, these compounds promiscuously inhibit a number of other transporters, including MCT1.

Novel potent inhibitors of MCT4 have been described, for example, in WO 2016/201426, the contents of which are hereby incorporated by reference in their entirety. The compound 2-([1-[2-(azetidin-1-yl)phenyl]-5-(3-cyclobutoxyphenyl)-1H-pyrazol-3-yl]methoxy)-2-methylpropanoic acid and related compounds have been described in WO 2018/111904, the contents of which are hereby incorporated by reference in their entirety, as inhibitors of MCT4 with promising potential.

There exists a need for new and improved methods for the synthesis of 2-([1-[2-(azetidin-1-yl)phenyl]-5-(3-cyclobutoxyphenyl)-1H-pyrazol-3-yl]methoxy)-2-methylpropanoic acid and related compounds which are amenable to large-scale synthesis.

Citation of any reference throughout this application is not to be construed as an admission that such reference is prior art to the present application.

### SUMMARY

Provided is a process for preparing a compound of Formula I

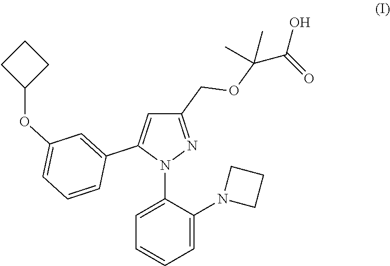 [SMILES: CC(C)(OCc1cc(-c2cccc(OC3CCC3)c2)n(-c2ccccc2N2CCC2)n1)C(=O)O]

or a salt thereof, comprising the steps of:
   i) contacting a compound of Formula II

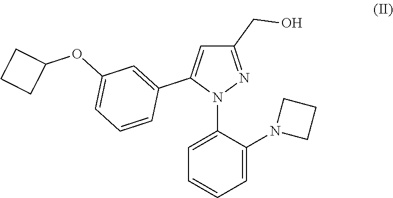 [SMILES: OCc1cc(-c2cccc(OC3CCC3)c2)n(-c2ccccc2N2CCC2)n1]

with a compound of Formula III

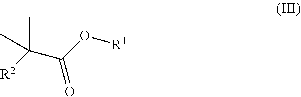

in a polar aprotic solvent in the presence of a non-nucleophilic base to produce a compound of Formula IV; and

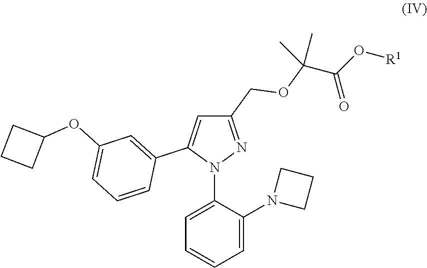

ii) hydrolyzing the compound of Formula IV to produce the compound of Formula I, or
   a salt thereof, wherein:
   R¹ is C₁-C₄ alkyl; and
   R² is halogen.

Also provided is a compound of formula

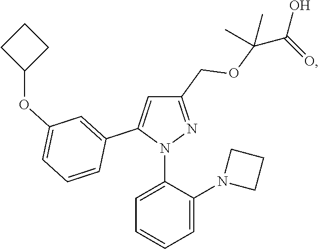 [SMILES: CC(C)(OCc1cc(-c2cccc(OC3CCC3)c2)n(-c2ccccc2N2CCC2)n1)C(=O)O]

prepared by the process described herein, wherein the compound contains a detectable amount that is less than 3% by weight of one or more residual organic solvents.

Also provided is a compound of formula

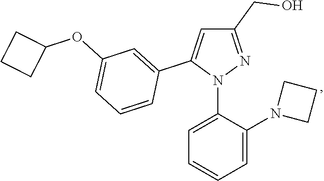 [SMILES: OCc1cc(-c2cccc(OC3CCC3)c2)n(-c2ccccc2N2CCC2)n1]

or a salt thereof.

Also provided is a compound of formula

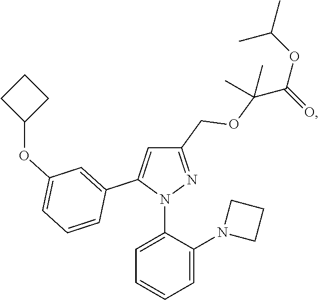 [SMILES: CC(C)OC(=O)C(C)(C)OCc1cc(-c2cccc(OC3CCC3)c2)n(-c2ccccc2N2CCC2)n1]

or a salt thereof.

Also provided herein is a method for treating a monocarboxylate transporter MCT4-mediated disorder in a subject in need thereof, comprising the step of administering to the subject therapeutically effective amounts of a compound described herein, or a salt thereof, or a composition described herein.

These and other aspects of the invention disclosed herein will be set forth in greater detail as the patent disclosure proceeds.

### DETAILED DESCRIPTION

Provided is a process for preparing a compound of Formula I

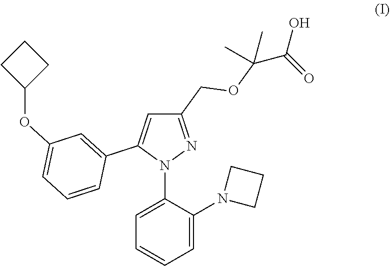 [SMILES: CC(C)(OCc1cc(-c2cccc(OC3CCC3)c2)n(-c2ccccc2N2CCC2)n1)C(=O)O]

or a salt thereof, comprising the steps of:
   i) contacting a compound of Formula II

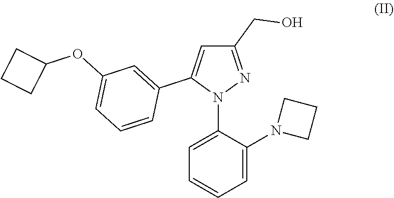 [SMILES: OCc1cc(-c2cccc(OC3CCC3)c2)n(-c2ccccc2N2CCC2)n1]

with a compound of Formula III

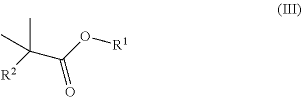

in a polar aprotic solvent in the presence of a non-nucleophilic base to produce a compound of Formula IV; and

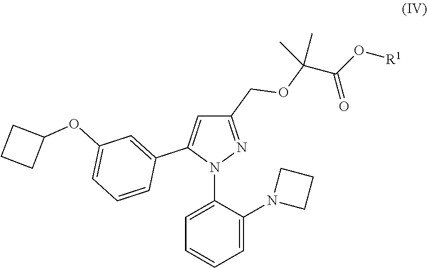

ii) hydrolyzing the compound of Formula IV to produce the compound of Formula I, or a salt thereof, wherein:
   R¹ is C₁-C₄ alkyl; and
   R² is halogen.

In some embodiments, R¹ is isopropyl and R² is bromine.

In some embodiments, the molar ratio of the compound of Formula III to the compound of Formula II is about 7.0 to about 10.0.

In some embodiments, the non-nucleophilic base is chosen from N,N-diisopropylethylamine (DIPEA), 8-diazabicycloundec-7-ene (DBU), 1,5-diazabicyclo(4.3.0)non-5-ene (DBN), 2,6-di-tert-butylpyridine, tert-butyl-lithium, tert-butyl-phosphazene, lithium diisopropylamide (LDA), sodium bis(trimethylsilyl)amide (NaHMDS), potassium tert-butoxide, potassium bis(trimethylsilyl)amide (KHMDS), lithium tetramethylpiperidide (LiTMP), sodium hydride, potassium hydride, and sodium tert-butoxide. In some embodiments, the non-nucleophilic base is potassium bis(trimethylsilyl)amide (KHMDS).

In some embodiments, the molar ratio of KHMDS to the compound of Formula II is about 4.0 to about 6.0.

In some embodiments, the polar aprotic solvent is chosen from acetone, acetonitrile, dichloromethane, dimethyl sulfoxide (DMSO) dimethylformamide (DMF), ethyl acetate, hexamethylphosphoric triamide (HMPT), pyridine, tetrahydrofuran (THF), and mixtures thereof.

In some embodiments, the polar aprotic solvent is a mixture comprised of THF and DMF.

In some embodiments, hydrolyzing the compound of Formula IV comprises reacting the compound of Formula IV with a nucleophilic base.

In some embodiments, the nucleophilic base is chosen from sodium hydroxide and sodium methoxide.

In some embodiments, the molar ratio of the nucleophilic base to the compound of Formula IV is about 18.0 to about 20.0.

In some embodiments, the compound of Formula II is prepared by contacting a compound of Formula V:

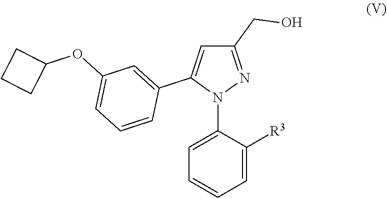

with azetidine in the presence of a palladium catalyst, wherein R³ is halogen.

In some embodiments, R³ is bromine.

In some embodiments, the molar ratio of azetidine to the compound of Formula V is about 2.0 to about 3.0.

In some embodiments, the molar ratio of palladium catalyst to the compound of Formula V is about 0.05 to about 0.15.

In some embodiments, the palladium catalyst is chosen from Pd(OAc)₂, palladium (II) pivalate, tetrakis(triphenylphosphine)palladium (0), bis(acetonitrile)palladium (II) dichloride, bis(triphenylphosphine)palladium (II) dichloride, [1,1′-bis(diphenylphosphino)ferrocene]palladium (II) dichloride, tris(dibenzylidenacetone)dipalladium (0), and palladium (II) chloride.

In some embodiments, the palladium catalyst is Pd(OAc)₂.

In some embodiments, the contacting is conducted in the presence of a ligand.

In some embodiments, the ligand is chosen from trimethylphosphine, triphenylphosphine, tricyclohexylphosphine, tri(o-tolyl)phosphine, 2-(dicyclohexylphosphino)-2′,4′,6′-tri-i-propyl-1,1′-biphenyl (XPhos), 2-(dicyclohexylphosphino)-2′-(N,N-dimethylamino) biphenyl, 2-dicyclohexylphosphino-2′,6′-di-i-propoxy-1,1′-biphenyl, 2-dicyclohexylphosphino-2′,6′-dimethoxy-1,1′-biphenyl, butyldi-1-adamantylphosphine, 2-(di-t-butylphosphino) biphenyl, 2-(dicyclohexylphosphino) biphenyl, (R)-(−)-1-[(S)-2-(diphenylphosphino) ferrocenyl]ethyldicyclohexylphosphine, 1,2-bis(diphenylphosphino)benzene (dppbenzene), 2,2′-bis(diphenylphosphino)-1,1′-binaphthyl (BINAP), bis(2-diphenylphosphinophenyl) ether (DPEphos), 4,5-bis(diphenylphosphino)-9,9-dimethylxanthene (Xantphos), 1,4-bis(diphenylphosphino) butane (dppb), 1,2-bis(diphenylphosphino) ethane (dppe), 1,1′-bis(diphenylphosphino)ferrocene (dppf), 1,3-bis(diphenylphosphino) propane (dppp) and [(t-Bu₃) PH]BF₄.

In some embodiments, the ligand is Xantphos.

In some embodiments, the molar ratio of ligand to the compound of Formula V is about 0.05 to about 0.15.

In some embodiments, the compound of Formula V is prepared by contacting a compound of Formula VI

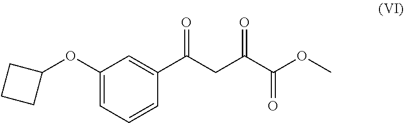 [SMILES: COC(=O)C(=O)CC(=O)c1cccc(OC2CCC2)c1]

with a compound of Formula VII:

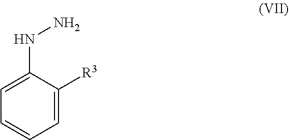

to form a compound of Formula VIII:

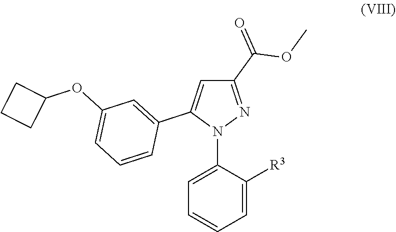

and subsequently contacting the compound of Formula VIII with a reducing agent to yield the compound of Formula V, wherein R³ is halogen.

In some embodiments, the reducing agent is chosen from lithium aluminium hydride, sodium bis(2-methoxyethoxy)aluminium hydride (Red-Al), nascent hydrogen, sodium amalgam, zinc amalgam, sodium borohydride, lithium borohydride, SmI₂, compounds containing the Fe²⁺ ion, compounds containing the Sn²⁺ ion, hydrazine, diisobutylaluminum hydride, and any combinations thereof.

In some embodiments, the reducing agent is sodium borohydride.

In some embodiments, the compound of Formula VI is prepared by contacting 1-(3-hydroxyphenyl)ethan-1-one with a compound of Formula IX:

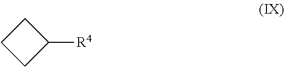

in the presence of a non-nucleophilic base in a polar aprotic solvent to form 1-(3-cyclobutoxyphenyl)ethan-1-one, which is subsequently contacted with dimethyl oxalate and a strong base to yield the compound of Formula VII, wherein R⁴ is chosen from halogen.

In some embodiments, the polar aprotic solvent is chosen from acetone, acetonitrile, dichloromethane, dimethyl sulfoxide (DMSO) dimethylformamide (DMF), ethyl acetate, hexamethylphosphoric triamide (HMPT), pyridine, tetrahydrofuran (THF), and mixtures thereof.

In some embodiments, the non-nucleophilic base is chosen from cesium carbonate, sodium carbonate, and potassium carbonate.

In some embodiments, the strong base is chosen from sodium hydroxide, sodium methoxide, sodium ethoxide, lithium diisopropylamide (LDA), sodium bis(trimethylsilyl)amide (NaHMDS), potassium tert-butoxide, potassium bis(trimethylsilyl)amide (KHMDS), lithium tetramethylpiperidide (LiTMP), sodium hydride, potassium hydride, and sodium tert-butoxide.

In some embodiments, the molar ratio of non-nucleophilic base to 1-(3-hydroxyphenyl)ethan-1-one is about 1.0 to about 2.0.

In some embodiments, the process further comprises converting the compound of Formula I to a tris salt of formula

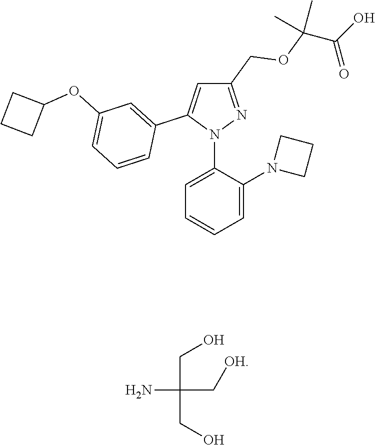 [SMILES: CC(C)(OCc1cc(-c2cccc(OC3CCC3)c2)n(-c2ccccc2N2CCC2)n1)C(=O)O ; NC(CO)(CO)CO]

Also provided is a tris salt prepared by a process as described herein.

Also provided is a compound of formula

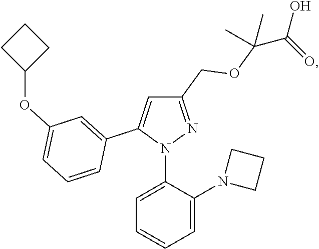 [SMILES: CC(C)(OCc1cc(-c2cccc(OC3CCC3)c2)n(-c2ccccc2N2CCC2)n1)C(=O)O]

prepared by a process described herein, wherein the compound contains a detectable amount, and in some embodiments, less than 3% by weight of residual organic solvent.

Also provided is a composition comprising at least 90%, such as at least 95%, 96%, 97%, 98%, 99%, or 99.5%, of a compound of Formula I, or a salt thereof, and a detectable amount of one or more impurities chosen from a compound of Formula IV, a compound of Formula II, a compound of Formula V, a compound of Formula VIII, 1-(3-hydroxyphenyl)ethan-1-one, bromocyclobutane, cesium carbonate, hydrochloric acid, 2-bromophenylhydrazine, sodium borohydride, xantphos, sodium tert-butoxide, isopropyl 2-bromo-2-methylpropanoate, potassium bis(trimethylsilyl)amide, sodium hydroxide, sodium hydride, sodium tert-pentoxide, acetonitrile, dichloromethane, 4-dimethylaminopyridine, ethyl acetate, dimethylformamide, ethanol, water, isopropanol, isopropyl acetate, potassium bis(trimethylsilyl)amide, methanol, methyl tert-butyl ether, sodium borohydride, N-methyl-2-pyrrolidone, palladium diacetate, sodium triacetoxyborohydride, tetrahydrofuran, and one or more heavy metals.

In some embodiments, the one or more heavy metals are chosen from platinum, palladium, iridium, rhodium, rhenium, ruthenium, cadmium, mercury, lead, arsenic, manganese, chromium, cobalt, nickel, copper, zinc, selenium, silver, antimony, thalium, nickel, vanadium, and zinc.

Also provided herein is a method for treating a monocarboxylate transporter MCT4-mediated disorder in a subject in need thereof, comprising the step of administering to the subject therapeutically effective amounts of a compound described herein or a composition described herein.

In some embodiments, the monocarboxylate transporter MCT4-mediated disorder is cardiac hypertrophy.

In some embodiments, the monocarboxylate transporter MCT4-mediated disorder is heart failure.

In some embodiments, the monocarboxylate transporter MCT4-mediated disorder is a cancer that expresses MCT4.

In some embodiments, the monocarboxylate transporter MCT4-mediated disorder is rheumatoid arthritis.

Also provided is a compound of formula

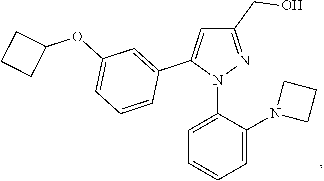 [SMILES: OCc1cc(-c2cccc(OC3CCC3)c2)n(-c2ccccc2N2CCC2)n1]

or a salt thereof.

Also provided is a compound of formula

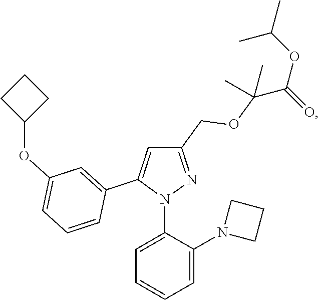 [SMILES: CC(C)OC(=O)C(C)(C)OCc1cc(-c2cccc(OC3CCC3)c2)n(-c2ccccc2N2CCC2)n1]

or a salt thereof.

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “alkyl,” as used herein, alone or in combination, refers to a straight-chain or branched-chain alkyl group containing from 1 to 20 carbon atoms. In certain embodiments, the alkyl will comprise from 1 to 10 carbon atoms. In further embodiments, the alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, nonyl and the like. The term “alkylene,” as used herein, alone or in combination, refers to a saturated aliphatic group derived from a straight or branched chain saturated hydrocarbon attached at two or more positions, such as methylene (—CH2-). Unless otherwise specified, the term “alkyl” may include “alkylene” groups.

The term “halo,” or “halogen,” as used herein, alone or in combination, refers to fluorine, chlorine, bromine, or iodine.

The term “non-nucleophilic base,” as used herein, refers to a sterically hindered organic base that is a poor nucleophile. Examples of non-nucleophilic bases include N,N-diisopropylethylamine (DIPEA), 8-diazabicycloundec-7-ene (DBU), 1,5-diazabicyclo(4.3.0)non-5-ene (DBN), 2,6-di-tert-butylpyridine, tert-butyl-lithium, tert-butyl-phosphazene, lithium diisopropylamide (LDA), sodium bis(trimethylsilyl)amide (NaHMDS), potassium tert-butoxide, potassium bis(trimethylsilyl)amide (KHMDS), lithium tetramethylpiperidide (LiTMP), sodium hydride, potassium hydride, sodium tert-butoxide, and potassium tert-butoxide. In certain embodiments, the non-nucleophilic base is potassium bis(trimethylsilyl)amide (KHMDS).

The term “nucleophilic base,” as used herein, means a Bronsted-Lowery base that is a good nucleophile. Examples include sodium methoxide, methyl lithium, sodium hydroxide, lithium hydroxide, sodium cyanide, potassium cyanide, sodium acetylide, sodium amide, sodium iodide, lithium bromide, potassium iodide, and sodium azide.

The term “polar solvent,” as used herein, refers to a solvent with large dipole moment.

The term “polar aprotic solvent,” as used herein, refers to a polar solvent that lacks an acidic hydrogen. Consequently, they are not hydrogen bond donors. Examples of polar aprotic solvents include acetone, acetonitrile, dichloromethane, dimethyl sulfoxide (DMSO) dimethylformamide (DMF), ethyl acetate, hexamethylphosphoric triamide (HMPT), pyridine, and tetrahydrofuran (THF). In certain embodiments, the polar aprotic solvent is THF, DMF, or a combination of THF and DMF.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reaction of the appropriate compound, in the form of the free base, with the appropriate acid.

The compounds disclosed herein can exist as polymorphs and other distinct solid forms such as solvates, hydrates, and the like. A compound may be a polymorph, solvate, or hydrate of a salt or of the free base or acid.

In some embodiments, the process further comprises formulating the product and/or a salt thereof into a pharmaceutical composition. The pharmaceutical composition may include one or more pharmaceutically-acceptable carriers. The pharmaceutical composition may be administered orally. The pharmaceutical composition may be delivered orally using tablets, troches, liquids, emulsions, suspensions, drops, capsules, caplets or gel caps and other methods of oral administration known to one skilled in the art. Suitable excipients for the pharmaceutical composition include one or more fillers, binders, and surfactants, glidants, lubricants, disintegrants, swelling agents, and antioxidants.

As used herein, the term, “detectable” refers to a measurable quantity measured using an HPLC method having a detection limit of 0.01 area-%.

As used herein, in connection with amounts of impurities, the term “not detectable” means not detected by the herein described HPLC method having a detection limit for impurities of 0.01 area-%.

As used herein, “limit of detection (LOD)” or “detection limit” refers to the lowest concentration of analyte that can be clearly detected above the base line signal, approximately three times the signal noise of the baseline.

Fillers include, but are not limited to, lactose, saccharose, glucose, starch, microcrystalline cellulose, microfine cellulose, mannitol, sorbitol, calcium hydrogen phosphate, aluminum silicate, amorphous silica, and sodium chloride, starch, and dibasic calcium phosphate dihydrate. In one embodiment, the filler is not water soluble, although it may absorb water. In one embodiment, the filler is a spheronization aid. Spheronization aids can include one or more of crospovidone, carrageenan, chitosan, pectinic acid, glycerides, β-cyclodextrin (β-CD), cellulose derivatives, microcrystalline cellulose, powdered cellulose, polyplasdone crospovidone, and polyethylene oxide.

Binders include, but are not limited to, cellulose ethers, methyl cellulose, ethyl cellulose, hydroxyethyl cellulose, propyl cellulose, hydroxypropyl cellulose, lower-substituted hydroxypropyl cellulose, hydroxypropylmethyl cellulose (hypromellose, e.g. hypromellose 2910, Methocel™ E), carboxymethyl cellulose, starch, pregelatinized starch, acacia, tragacanth, gelatin, polyvinyl pyrrolidone (povidone), cross-linked polyvinyl pyrrolidone, sodium alginate, microcrystalline cellulose, and lower-alkyl-substituted hydroxypropyl cellulose. In one embodiment, the binders are selected from wet binders.

Surfactants include, but are not limited to, anionic surfactants, including sodium lauryl sulfate, sodium deoxycholate, dioctyl sodium sulfosuccinate, and sodium stearyl fumarate, nonionic surfactants, including polyoxyethylene ethers, and polysorbate 80, and cationic surfactants, including quaternary ammonium compounds. In one embodiment, the surfactant is selected from anionic surfactants, e.g. sodium lauryl sulfate.

Disintegrants include, but are not limited to, starch, sodium cross-linked carboxymethyl cellulose, carmellose sodium, carmellose calcium, cross-linked polyvinyl pyrrolidone, and sodium starch glycolate, low-substituted hydroxypropyl cellulose, and hydroxypropyl starch.

Glidants include, but are not limited to, polyethylene glycols of various molecular weights, magnesium stearate, calcium stearate, calcium silicate, fumed silicon dioxide, magnesium carbonate, magnesium lauryl sulfate, aluminum stearate, stearic acid, palmitic acid, cetanol, stearol, and talc.

Lubricants include, but are not limited to, stearic acid, magnesium stearate, calcium stearate, aluminum stearate, and siliconized talc.

In certain embodiments, the formulation further comprises one or more antioxidants. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

### METHODS

### General Synthetic Methods for Preparing Compounds

The following invention is further illustrated by the following Examples.

In the Examples below and throughout the disclosure, the following abbreviations may be used: DMF=dimethylformamide; KHMDS=potassium bis(trimethylsilyl)amide; MTBE=methyl tert-butyl ether=TBME=tert-butyl methyl ether; NMP=N-methyl-2-pyrrolidone; THF=tetrahydrofuran; ¹H-NMR=Proton Nuclear magnetic Resonance; ICP=Inductively Coupled Plasma mass spectrometry; IPC=In Process Control/Check. Other abbreviations may be used and will be familiar in context to those of skill in the art.

### Example 1: Synthesis of 2-([1-[2-(azetidin-1-yl)phenyl]-5-(3-cyclobutoxyphenyl)-1H-pyrazol-3-yl]methoxy)-2-methylpropanoic acid

### 1. Synthesis of Compound 7

Compound 7 was synthesized by the process of Scheme 1.

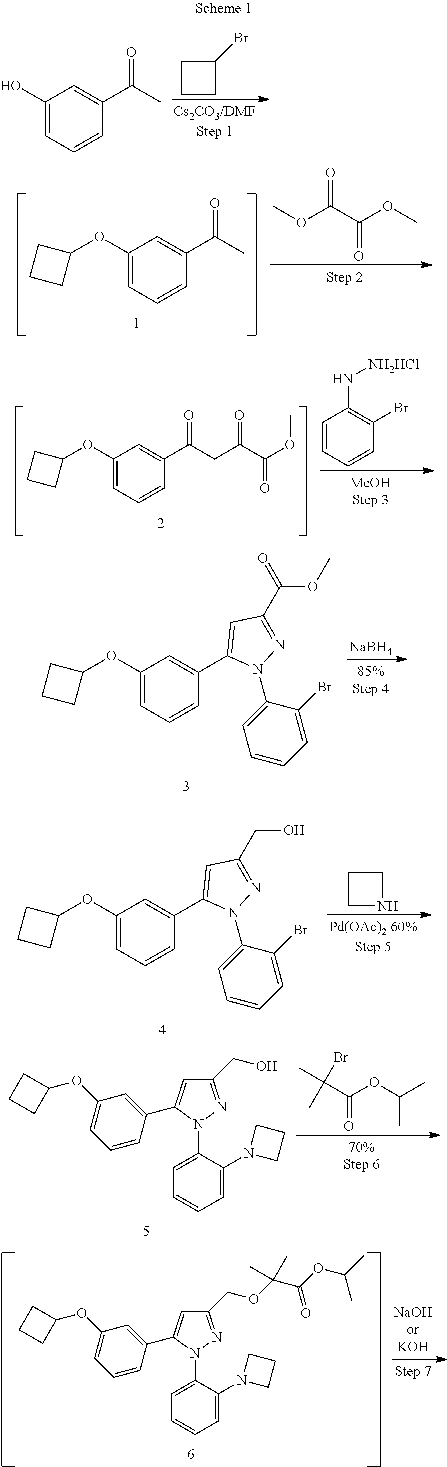

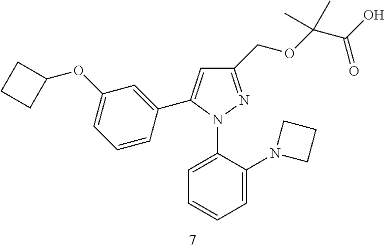 [SMILES: CC(C)(OCc1cc(-c2cccc(OC3CCC3)c2)n(-c2ccccc2N2CCC2)n1)C(=O)O]

### Step 1:

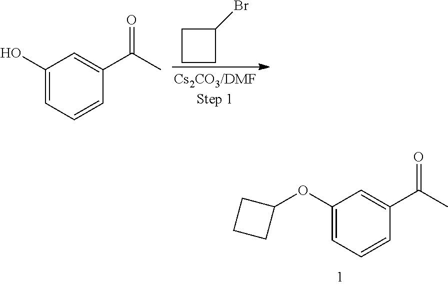

A mixture of 1-(3-hydroxyphenyl)ethan-1-one (20 kg), bromocyclobutane (1.3 eq.) and Cs₂CO₃ (1.5 eq.) in DMF (5 volumes) was stirred for 16 hours at 80±5° C. The reaction vessel was charged with water (15 volumes) and methyl tert-butyl ether (MTBE, 15 volumes). The organic layer was separated and washed twice with 20% brine (5 volumes), then concentrated until about 5 volumes and then solvent-switched with methanol three times until about 5 volumes. The crude methanolic solution containing Compound 1 was used without further purification in the next step.

### Step 2:

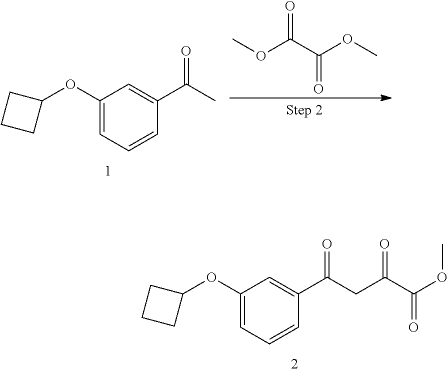

To the crude methanolic solution of Step 1 (5 volumes) was added sodium methoxide (2.0 eq.) and dimethyl oxalate (1.5 eq.). The reaction vessel was stirred for 16 hours at 30±5° C. Upon completion, the reaction mixture was cooled to a temperature between 0-10° C. The pH was adjusted to 2-3 with 4.0 M HCl in methanol, and the crude solution containing Compound 2 was used without further purification in the next step.

### Step 3:

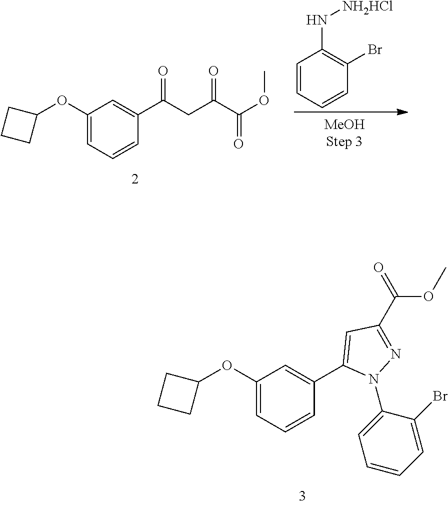

To the reaction vessel containing the crude methanolic solution of Step 2 was added 2-bromophenylhydrazine hydrochloride (1.0 eq.). The reaction mixture was then stirred for 10 hours at 60±5° C., then cooled to 5-15° C. The resulting solids were filtered, washed with methanol (1 volume), and then slurried with water (20 volumes). The solids were filtered again and further washed with water (2 volumes). The product was dried under 50° C. in an oven to afford 54.9 kg of Compound 3 (88% yield for steps 1-3).

### Step 4:

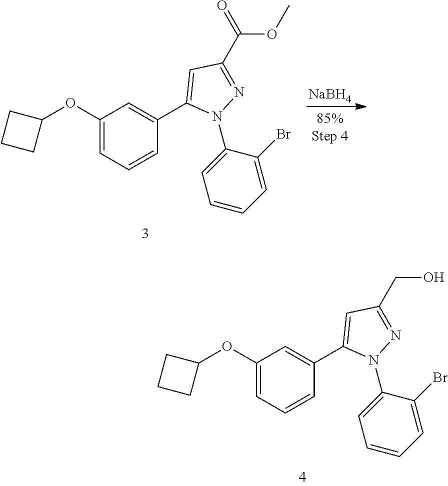 [SMILES: COC(=O)c1cc(-c2cccc(OC3CCC3)c2)n(-c2ccccc2Br)n1 ; "NaBH4" ; OCc1cc(-c2cccc(OC3CCC3)c2)n(-c2ccccc2Br)n1]

Compound 3 (4.0 kg; 1.0 eq.) was added to THF (4 volumes) before methanol (0.4 vol.) was further added to the reaction vessel which was then cooled to 10-25° C. Sodium borohydride (1.2 eq) was added, and the reaction was stirred for 16 hours at 10-25° C. Upon completion, the reaction was cooled to 0-10° C., and the pH was adjusted to 3-5 with 0.5 M HCl, at which point, the product precipitated out of solution. The mixture was stirred for an additional 1-2 hours at 5-15° C. The product was collected via filtration, washed with water, and dried under 50° C. in an oven to afford 3.5 kg of Compound 4, 85% yield.

Step 4 was repeated at 5× scale with Compound 3 (20.0 kg; 1.0 eq.) added to THF (6.0 volumes) before methanol (0.4 vol.) was further added to the reaction vessel which was then cooled to 25±5° C. Sodium borohydride (1.2 eq) was added, and the reaction was stirred for 16 hours at 25±5° C. Upon completion, the reaction was cooled to 0-10° C., and the pH was adjusted to 3-5 with 0.5 M HCl, at which point, the product precipitated out of solution. The mixture was stirred for an additional 1-2 hours at 5-15° C. The product was collected via filtration, washed with water, and dried under 50° C. in an oven to afford 18.2 kg of Compound 4 at 99.4% purity and an isolated yield of 97%.

### Step 5:

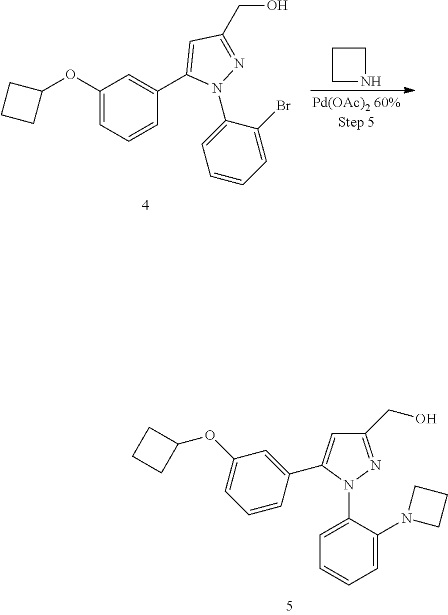

Compound 4 (2.97 kg; 1.0 eq.) was added with stirring and under a nitrogen atmosphere to THF (10 volumes). Xantphos (0.11 eq.) and Pd(OAc)₂ (0.11 eq.) and t-BuONa (2.0 eq.) were added to the reaction vessel. Azetidine (2.5 eq.) was added, and the resulting mixture was stirred for 20 hours at 25-35° C. The vessel was charged with MTBE (20 volumes) and water (20 volumes). The organic layer was separated, washed with 5% aqueous NH₄Cl and 5% aqueous NaCl and filtered through Celite®. The resulting organic layer was concentrated and then solvent-switched with ethyl acetate (10 volumes). The resulting solution was stirred for 303 minutes at 70-80° C., then cooled to 15-20° C. and stirred for an additional 1-2 hours. The solids were collected via filtration, washed with ethyl acetate (0.5 volumes), and dried under 50° C. in an oven to afford 1.7 kg of Compound 5, 60% yield. When reproduced with 18.0 kg of Compound 4, 10.8 kg of Compound 5 were afforded, with a purity of 98.8% and an isolated yield of 63.8%.

### Step 6

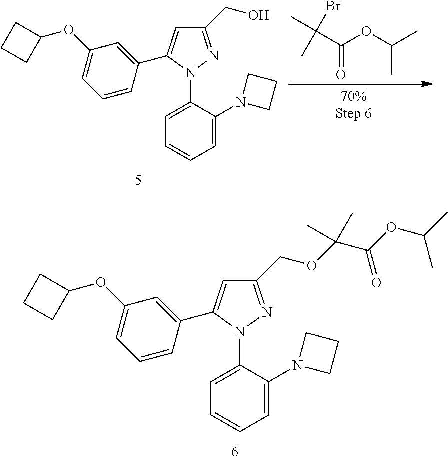

To a solution of Compound 5 (700.0 g, 1 eq.) in DMF (8 volumes) was added isopropyl 2-bromo-2-methylpropanoate (8.0 eq.). The reaction mixture was cooled to between-60° C. and −50° C. using liquid nitrogen. A THE solution of potassium bis(trimethylsilyl)amide (KHMDS, 5.0 eq.) was added dropwise over 1.5 hours; then the reaction was stirred for 30-50 minutes. Water (10 volumes) was added to the reaction vessel. The organic phase was collected, washed with NaCl (15%) (5 volumes) twice, and then concentrated. The crude Compound 6 (70% yield) was used directly in the next step without further purification.

### Step 7

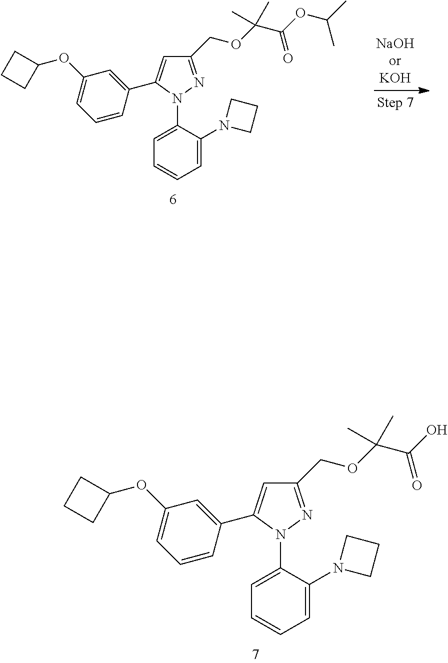 [SMILES: CC(C)OC(=O)C(C)(C)OCc1cc(-c2cccc(OC3CCC3)c2)n(-c2ccccc2N2CCC2)n1 ; CC(C)(OCc1cc(-c2cccc(OC3CCC3)c2)n(-c2ccccc2N2CCC2)n1)C(=O)O]

Crude Compound 6 (1950.0 g, 1 eq.) from step 6 was added to the reaction vessel with 30% (aq.) NaOH (10 volumes, 20 eq.) and methanol (5 volumes). The reaction was stirred for 16 hours at 50±5° C., then cooled to 15-30° C. The solution was washed twice with MTBE (10 volumes), then the aqueous layer was separated and filtered through Celite®. The pH of the resulting aqueous phase was adjusted to 3-5 using 2.0 M HCl. The solution was cooled to 5-10° C. and stirred for 2-3 hours, then filtered. The filter cake was dissolved in THF (10 volumes), then mercapto silica gel (300 g, 15% w/w) was added to the solution. The resulting mixture was heated to 50-60° C., stirred for 3 hours, and then filtered. The solution was concentrated, then the crude product was dissolved in ethyl acetate (10 volumes). The resulting solution was heated to 70-80° C. and stirred for 3-4 hours, then cooled to 0-10° C. The solids were collected via filtration, washed with ethyl acetate (0.5 volumes), then dried under 50° C. in an oven to afford Compound 7 (1.4 kg, 58% yield) as an off-white solid.

This reaction was repeated at a larger scale with crude Compound 6 (8.4 kg, 1 eq.) from step 6 was added to the reaction vessel with 30% (aq.) KOH (10 volumes, 20 eq.) and methanol (5 volumes). After following the same workup as above, 8.76 kg of Compound 7 were afforded at a purity of 97.7% and a yield of 67% over two steps.

### 2. Use of Different Propanoate Esters in Step 6

Varying the propanoate ester in step 6 was explored. The results are shown in Table 1.

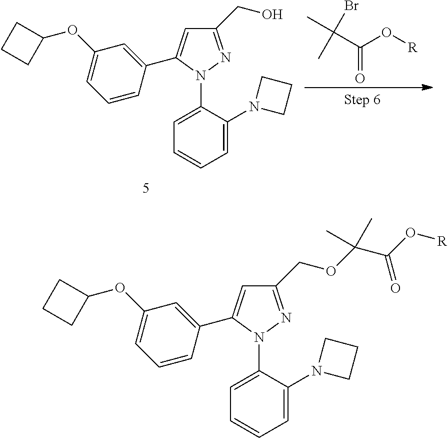

TABLE 1

| | | | Reaction | | |
|---|---|---|---|---|---|
| | Starting Materials and | | Conditions | | IPC |
| Trial | Reagents | | Temp. | Time | Starting |
| # | R | Reagents | ° C. | (h) | Material:Product |
| 1 | Methyl | NaH (4.0 eq.) | 0~15 | 16 | 6.0%:82.5% |
| | | DMF (10.0 v) | | | |
| 2 | Methyl | NaH (4.5 eq.) | 0~10 | 18 | 6.7%:46.2% |
| | | DME (10.0 v) | | | |
| 3 | Methyl | NaH (4.5 eq.) | 0~10 | 18 | 9.9%:62.9% |
| | | NMP (10.0 V) | | | |
| 4 | Methyl | tBuONa (4.0 eq.) | 0~15 | 16 | 15.9%:39.5% |
| | | DMF (10.0 V) | | | |
| 5 | Methyl | Sodium tert- | 0~15 | 16 | 19.9%:40.7% |
| | | pentoxide (4.0 eq.) | | | |
| | | DMF (10.0 V) | | | |
| 6 | Methyl | Cs₂CO₃ (6.0 eq.) | 20~30 | 18 | 83.5%:11.6% |
| | | DMF (10.0 V) | | | |
| 7 | Methyl | NaI(0.1 eq.) | 50~55 | 18 | 47.0%:37.7% |
| | | Cs₂CO₃(6.0 eq.) | | | |
| | | DMF (10.0 V) | | | |
| 8 | Methyl | Cs₂CO₃ (6.0 eq.) | 80~90 | 36 | 34.4%:33.7% |
| | | DMF (10.0 V) | | | |
| 9 | t-Butyl | NaH (4.0 eq.) | 0~15 | 18 | 9.9%:34.7% |
| | | DMF (10.0 V) | | | |
| 10 | t-Butyl | tBuONa (4.0 eq.) | 0~15 | 18 | 33.0%:26.0% |
| | | DMF (10.0 V) | | | |
| 11 | t-Butyl | Sodium tert- | 0~15 | 18 | 36.4%:25.5% |
| | | pentoxide (4.0 eq.) | | | |
| | | DMF (10.0 V) | | | |
| 12 | Methyl | LiHMDS (4.5 eq.) | 0~10 | ~18 | 98.2%:0% |
| | | THF (10.0 v) | | | |
| 13 | Methyl | KHMDS (1.0 eq.) | −50~−40 | 1.5~2 | 31.5%:45.0%* |
| | | DMF (10.0 v) | | | |
| 14 | Isopropyl | KHMDS (6.0 eq.) | −60~−50 | 1.5~2 | 4.9%:84.5% |
| | | DMF (20.0 V) | | | |
| 15 | Isopropyl | DMF (20.0 V) | −60~−50 | Add 5 | 52.6%:38.3% |
| | | KHMDS (1.2 eq.) | | mins + | |
| | | | | Stir | |
| | | | | 15~20 | |
| | | | | mins | |
| 16 | Isopropyl | KHMDS (1.2 eq.), | −60~−50 | Add 5 | 16.8%:72.3% |
| | | Total 2.4 eq. | | mins+ | |
| | | | | Stir | |
| | | | | 15~20 | |
| | | | | mins | |
| 17 | Isopropyl | KHMDS (1.2 eq.), | −60~−50 | Add 5 | 6.2%:82.5% |
| | | Total 3.6 eq. | | mins + | |
| | | | | Stir | |
| | | | | 15~20 | |
| | | | | mins | |
| 18 | Isopropyl | KHMDS (1.2 eq.), | −60~−50 | Add 5 | 4.5%:88.3% |
| | | Total 4.8 eq. | | mins + | |
| | | | | Stir | |
| | | | | 15~20 | |
| | | | | mins | |
| 19 | Isopropyl | KHMDS (1.2 eq.), | −60~−50 | Add 5 | 3.2%:89.6% |
| | | Total 6.0 eq. | | mins + | |
| | | | | Stir | |
| | | | | 15~20 | |
| | | | | mins | |

*Additionally, 13.4% of a dimer side product was detected upon completion of the reaction.

As shown in Table 1, the starting material with the isopropyl ester performed better than the t-butyl ester and methyl ester. Additionally, use of the isopropyl ester limits the potential formation of a dimer (or bis-adduct) side product, which was detected when using the methyl ester in Trial 13. Use of the isopropyl ester also permits the use of a solvent other than DMF/NaH. The reaction also proceeded much more quickly than with DMF/NaH.

### 3. Formation of Tris Salt of Compound 7

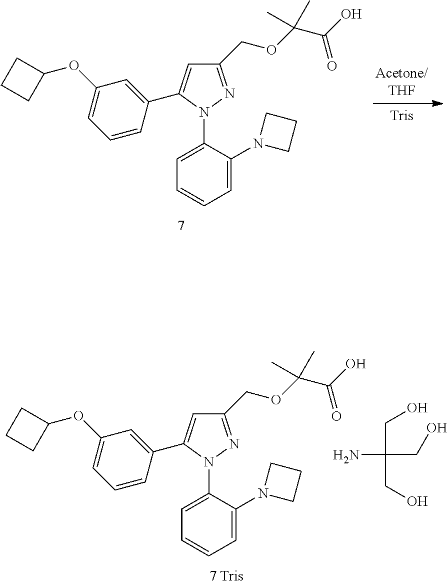

Compound 7 (8.4 kg, 1 eq.) was added to a reaction vessel with THF (42 L, 5.0 volumes), charged with mercapto silica gel (420 g, 5 wt. %), and stirred and heated at 50-60° C. for three hours. The reaction mixture was sampled and concentrated for ICP analysis to determine the residual Pd concentration (˜15 ppm). The reaction mixture was filtered charged with acetone (168 L, 20.0 volumes) and tris(hydroxymethyl)aminomethane (“Tris”, 1.01 eq.) solution in water (4.2 L, 0.5 volumes). The reaction mixture was stirred for 5-10 minutes at 20-30° C. for most of the solids to dissolve, and the continued to stir for about 20 hours at 20-30° C. to permit the Tris salt of Compound 7 to precipitate out. The precipitant was filed, washed with acetone (4.2 L, 0.5 volumes) and dried under vacuum at 60° C. to afford 8.4 kg of Compound 7 Tris at 99.5% purity and a yield of 79.2%, having 1690 ppm residual acetone and 962 ppm residual THF.

### Example 2: Pharmacokinetic Assays

The tris salt of the compound of Formula I and the free acid were tested in two animal models to assess pharmacokinetic parameters.

### Rat Model

Groups, Dosing, and Collection. The pharmacokinetics of the free acid and the tris salt of the compound of Formula I by oral gavage were assessed in male Sprague-Dawley rats. Three rats per group were perorally administered 20, 60, or 200 mg free acid or 25, 75, or 250 mg in 0.5% methylcellulose in saline for a final concentration of 2, 6, or 20 mg/mL free acid or 2.5, 7.5, or 20 mg/mL. Plasma was collected at 5 min, 15 min, 30 min, 1 hour, 2 hour, 4 hour, 6 hour, 8 hour, and 24 hour post dose via jugular vein. No abnormal clinical symptoms were observed.

Stock and Dose Prep. Stock solutions were prepared by dissolving 2.47 mg of the compound of Formula I (free acid) in 2.470 mL DMSO with vortexing to obtain 1 mg/mL solution of free acid, or 2.09 mg of Tris salt in 1.655 mL DMSO with vortexing to obtain 1 mg/mL solution of Tris salt. Dosing solutions were prepared by vortexing/sonicating the following solids in solvent:

TABLE 2
Preparation of Dosing Solutions

| Dose, | | |
|---|---|---|
| mg/mL | Solid | Solvent |
| 2 | 40.1 mg free acid | 20.050 mL 0.5% MC in saline |
| 6 | 119.26 free acid | 19.877 mL 0.5% MC in saline |
| 20 | 396.69 free acid | 19.835 mL 0.5% MC in saline |
| 2.5 | 62.94 tris salt | 19.942 mL 0.5% MC in saline |
| 7.5 | 180.32 tris salt | 19.044 mL 0.5% MC in saline |
| 25 | 595.39 tris salt | 18.864 mL 0.5% MC in saline |

LC MS-MS Analysis. Liquid Chromatography with tandem mass spectrometry was used to determine plasma concentrations of the compound of Formula I free acid and the compound of Formula I tris salt in plasma samples collected at prescribed time points.

Appropriate serial concentrations of working solutions were achieved by diluting a stock solution of analyte with 50% acetonitrile in water solution. Five μL of working solutions (10, 20, 50, 100, 500, 1000, 5000, 8000, 10000 ng/mL) were added to 50 μL of the blank male SD Rat plasma to achieve calibration standards of 1˜1000 ng/ml (1, 2, 5, 10, 50, 100, 500, 800, 1000 ng/ml) in a total volume of 55 μL. Five quality control (QC) samples at 2 ng/mL, 5 ng/ml, 10 ng/ml, 50 ng/ml and 800 ng/mL for plasma were prepared independently of those used for the calibration curves. These QC samples were prepared on the day of analysis in the same way as calibration standards. 55 μL standards, 55 μL QC samples and 55 μL unknown samples (50 μL plasma/blood with 5 μL blank solution) were added to 200 μL of methanol containing internal standard (dexamethasone) mixture for precipitating protein, respectively, and vortexed for 30 seconds. After centrifugation at 4° C. and 4000 rpm for 15 minutes, the supernatant was diluted with water with a ratio of 1:2. 2 μL of the supernatant was injected into the LC/MS/MS system for quantitative analysis.

Instrumentation included HALO 90A C18 2.7 μm 2.1×50 mm HPLC column, a Prominence degasser DGU-20A5R(C), liquid chromatograph Shimadzu LC-30AD with communications bus module CBM-20A and Auto SIL-20AC HT; and an AB Sciex Triple Quad 5500 LC/MS/MS instrument. The following conditions were used:

| Mobile Phase | | |
|---|---|---|
| Solution A: | 5% Acetonitrile in Water (0.1% Formic acid) | |
| Solution B: | 95% Acetonitrile in Water (0.1% Formic acid) | |
| Gradient | | |
| Flow rate: | 0.5 mL/min | |
| Time (min) | A (%) | B (%) |
| 0.20 | 95.0 | 5.00 |
| 1.50 | 0.00 | 100 |
| 2.00 | 0.00 | 100 |
| 2.01 | 95.0 | 5.00 |
| 2.50 | 95.0 | 5.00 |

| | |
|---|---|
| Injection volume: | 2 μL |

Results. Results are given below in Table 3 (free acid) and Table 4 (tris salt). Note that because the formula weight is higher for the tris salt (FW=582.7) than for the free acid (MW=461.6), the appropriate comparison is 20 mg/kg free acid to 25 mg/kg tris salt. Overall, the total drug exposure across time (AUCₗₐₛₜ and AUC_{Inf}) was higher in both male and female subjects in the tris salt groups than in the free acid groups. Maximum exposure levels (Cₘₐₓ) were also higher in both male and female subjects in the tris salt groups than in the free acid groups, with the exception of females dosed at 20/25 mg/kg, where the free acid Cₘₐₓ (78,883 ng/mL) was slightly higher than the tris salt Cₘₐₓ (68,567 ng/mL). In general, exposure was higher in female subjects than male subjects.

TABLE 3
PK Parameters, Free Acid

| | 20 mg/kg, | 60 mg/kg, | 200 mg/kg, | 20 mg/kg, | 60 mg/kg, | 200 mg/kg, |
|---|---|---|---|---|---|---|
| | male: mean | male: mean | male: mean | female: mean | female: mean | female: mean |
| PK parameter | (SD, CV %) | (SD, CV %) | (SD, CV %) | (SD, CV %) | (SD, CV %) | (SD, CV %) |
| T_{1/2}, h | 3.27 | 2.92 | 3.14 | 7 | 9.1 | 10.4 |
| | (1.05, 32.3) | (0.42, 14.6) | (0.34, 10.8) | (1.71, 24.5) | (5, 54.6) | (3.6, 34.8) |
| Tₘₐₓ, h | 1 | 2.67 | 4 | 2.67 | 4.67 | 4.67 |
| | (0, 0) | (1.15, 43.3) | (2, 50) | (1.15, 43.3) | (2.31, 49.5) | (1.15, 24.7) |
| Cₘₐₓ, ng/mL | 30633 | 56500 | 114533 | 78833 | 113000 | 206000 |
| | (2954, 9.64) | (8805, 15.6) | (19433, 17) | (8456, 10.7) | (15716, 13.9) | (25981, 12.6) |
| AUCₗₐₛₜ, | 166649 | 445134 | 1104950 | 884621 | 1425555 | 2852952 |
| h*ng/mL | (35652, 21.4) | (129431, 29.1) | (348568, 31.5) | (206245, 23.3) | (127537, 8.95) | (613410, 21.5) |
| AUC_{Inf}, | 167534 | 447474 | 1113663 | 990680 | 1770576 | 3815512 |
| h*ng/mL | (34931, 20.9) | (131210, 29.3) | (354633, 31.8) | (280475, 28.3) | (506147, 28.6) | (1390909, 36.5) |
| AUC_{_{—}}_{% Extrap}_obs, % | 0.61 | 0.477 | 0.704 | 9.9 | 16.7 | 22.1 |
| | (0.68, 110) | (0.313, 65.7) | (0.406, 57.6) | (5.6, 56.8) | (14.6, 87.2) | (13.2, 59.8) |
| MRT_{Inf}_obs, h | 4.54 | 5.33 | 6.01 | 10 | 13.6 | 15.9 |
| | (0.49, 10.9) | (0.66, 12.4) | (1.34, 22.4) | (2.4, 23.9) | (6.7, 49.3) | (6.1, 38.4) |
| AUCₗₐₛₜ/D, | 8332 | 7419 | 5525 | 44231 | 23759 | 14265 |
| h*ng/mL | (1783, 21.4) | (2157, 29.1) | (1743, 31.5) | (10312, 23.3) | (2126, 8.95) | (3067, 21.5) |

TABLE 4
PK Parameters, Tris Salt

| | 25 mg/kg, | 75 mg/kg, | 250 mg/kg, | 25 mg/kg, | 75 mg/kg, | 250 mg/kg, |
|---|---|---|---|---|---|---|
| | male: mean | male: mean | male: mean | female: mean | female: mean | female: mean |
| PK parameter | (SD, CV %) | (SD, CV %) | (SD, CV %) | (SD, CV %) | (SD, CV %) | (SD, CV %) |
| T_{1/2}, h | 3.24 | 2.91 | 12.2 | 6.53 | 9.6 | 40.5 |
| | (1.26, 39) | (0.36, 12.2) | (5.5, 45.2) | (0.76, 11.6) | (5.3, 55.1) | (40, 98.8) |
| Tₘₐₓ, h | 2.86 | 4 | 5.33 | 6 | 5.33 | 6.67 |
| | (4.46, 156) | (2, 50) | (1.15, 21.7) | (0, 0) | (2.31, 43.3) | (1.15, 17.3) |
| Cₘₐₓ, ng/mL | 44233 | 92300 | 143667 | 68567 | 171333 | 223000 |
| | (12644, 28.6) | (14722, 15.9) | (26951, 18.8) | (6405, 9.3) | (11590, 6.76) | (29866, 13.4) |
| AUCₗₐₛₜ, | 351930 | 905824 | 2085035 | 947420 | 2541137 | 4467175 |
| h*ng/mL | (139125, 39.5) | (39934, 4.41) | (193187, 9.27) | (135087, 14.3) | (502767, 19.8) | (704885, 15.8) |
| AUC_{Inf}, | 356683 | 910102 | 2888539 | 1044472 | 3274996 | 16320220 |
| h*ng/mL | (140112, 39.3) | (39184, 4.31) | (359261, 12.4) | (173985, 16.7) | (1267800, 38.7) | (14774911, 90.5) |
| AUC_{_{—}}_{% Extrap}_obs, % | 1.28 | 0.473 | 26.5 | 9 | 18.5 | 59.9 |
| | (1.78, 139) | (0.225, 47.7) | (16.5, 62.2) | (2.5, 27.6) | (15.9, 85.7) | (21.2, 35.5) |
| MRT_{Inf}_obs, h | 6.1 | 6.03 | 18.6 | 9.9 | 14.7 | 62 |
| | (1.3, 21.3) | (0.39, 6.51) | (7.6, 40.8) | (1.1, 11.35) | (7.6, 52) | (56, 90.4) |
| AUCₗₐₛₜ/D, | 14077 | 12078 | 8340 | 37897 | 33882 | 17869 |
| h*ng/mL | (5565, 39.5) | (532, 4.41) | (773, 9.27) | (5403, 14.3) | (6704, 19.8) | (2820, 15.8) |

### Dog Model

Groups, Dosing, and Collection. The pharmacokinetics of the free acid and the tris salt of the compound of Formula I administered by oral capsules was assessed in dogs. Three male beagle dogs were administered a single dose of 3 mg/kg free acid on Day 1 of the study, and a single dose of 3 mg/kg tris salt on Day 8 of the study. Plasma was collected pre-dose, and at 0.5, 1, 2, 4, 8, 12 and 24 hours post-dose via puncture of peripheral veins. No abnormal clinical symptoms were observed.

In a follow-up study, the pharmacokinetics of tris salt of the compound of Formula I were investigated via oral gavage of a 0.5% methylcellulose in saline formulation. Two groups of animals were tested. In Group 1, three male beagle dogs were administered a single dose of 3 mg/kg tris salt. In Group 2, three male beagle dogs were administered a single dose of 30 mg/kg tris salt. Plasma was collected pre-dose, and at 0.5, 1, 2, 4, 8, 12 and 24 hours post-dose via puncture of peripheral veins. No abnormal clinical symptoms were observed.

LC MS-MS Analysis. Liquid Chromatography with tandem mass spectrometry was used to determine plasma concentrations of the compound of Formula I free acid and the tris salt in plasma samples collected at prescribed time points. Instrumentation included YMC-Triart C₁₈, S-5 μm (50×2.1 mm) HPLC column, liquid chromatograph Shimadzu LC-30AD with communications bus module CBM-20A and Auto SIL-20AC HT; and a Triple Quad 5500 LC/MS/MS instrument. Tolbutamide was used as an internal standard. The following conditions were used:

| | |
|---|---|
| HPLC Conditions: | |
| Mobile phases: | A: 0.1% Formic acid in DI water; |
| | B: 0.1% Formic acid in Acetonitrile |
| Injection | 20 μl |
| volume: | |
| Autosampler | 4° C. |
| temp.: | |
| Retention time: | Both free acid and salt: |
| | 1.37 min; tolbutamide 1.22 min |
| Gradient | |
| Flow Rate: | 600 (μL/min) |

| Time (minute) | A (%) | B (%) |
|---|---|---|
| 0.50 | 90 | 10 |
| 0.80 | 10 | 90 |
| 1.80 | 10 | 90 |
| 2.00 | 90 | 10 |
| 2.50 | 90 | 10 |

Mass Spectrometer Conditions:

| Ionization | Negative | | | | | |
|---|---|---|---|---|---|---|
| mode: | | | | | | |
| Monitoring | MRM | | | | | |
| mode: | | | | | | |
| Capillary | −4500 | | | | | |
| (volts): | | | | | | |
| Gas | 550° C. | | | | | |
| Temperature: | | | | | | |
| | Transition | Dwell | DP | CE | EP | CXP |
| Compound | (m/z) | Time (ms) | (v) | (v) | (v) | (v) |
| Cpd. 1, Free | 460.3→432.1 | 100 | −134 | −37 | −10 | −14 |
| Acid/Salt | | | | | | |
| Tolbutamide | 269.0→169.9 | 100 | −75 | −22 | −10 | −1 |

Results. Results from the powder in capsule are given below in Table 5. Results from oral gavage of the suspension are given below in Table 6. Overall, the total drug exposure across time (AUCₗₐₛₜ and AUC_{Inf}) was higher for the tris salt (both powder in capsule and suspension formulations) compared to the free acid. Half-life appeared lower for tris salt compared to free acid, but mean residence time increased. One animal in the salt powder in capsule experiment showed abnormally high plasma concentration at the first two timepoints (30-50× the other two subjects), so SD and CV % were not calculated.

TABLE 5
PK parameters in dogs tested with
the powder in capsule formulation

| | Powder in Capsule | | | | | |
|---|---|---|---|---|---|---|
| | Free Acid | | | Salt | | |
| | Mean | SD | CV % | Mean | SD | CV % |
| T_{1/2} | 6.33 | 0.736 | 11.6 | 3.63 | — | — |
| AUCₗₐₛₜ | 1215 | 323 | 26.6 | 3924 | — | — |
| AUC_{inf} | 1265 | 335 | 26.5 | 4092 | — | — |
| MRTₗₐₛₜ | 5.39 | 0.796 | 14.8 | 7.89 | — | — |
| Cl_{F} | 2505 | 761 | 30.4 | 756 | — | — |

TABLE 6
PK parameters in dogs tested with the oral suspension

| | Suspension | | | | | |
|---|---|---|---|---|---|---|
| | 3 mg/kg | | | 30 mg/kg | | |
| | Mean | SD | CV % | Mean | SD | CV % |
| T_{1/2} | 4.74 | 3.31 | 69.8 | 3.69 | — | — |
| AUCₗₐₛₜ | 5075 | 1333 | 26.3 | 431317 | 27909 | 67.5 |
| AUC_{inf} | 5202 | 1359 | 26.1 | 56792 | — | — |
| MRTₗₐₛₜ | 2.34 | 0.655 | 28.0 | 3.81 | 3.24 | 85.0 |
| Cl_{F} | 602 | 146 | 24.3 | 547 | — | — |

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

We claim:

1. A process for preparing a compound of Formula I 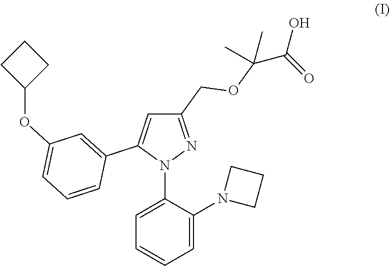 [SMILES: CC(C)(OCc1cc(-c2cccc(OC3CCC3)c2)n(-c2ccccc2N2CCC2)n1)C(=O)O]
or a salt thereof, comprising the steps of:
i) contacting a compound of Formula II 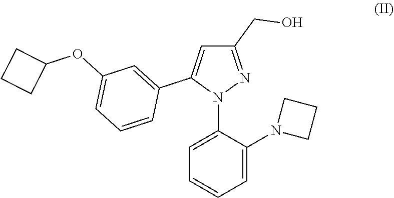 [SMILES: OCc1cc(-c2cccc(OC3CCC3)c2)n(-c2ccccc2N2CCC2)n1]
with a compound of Formula III 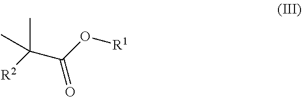
in a polar aprotic solvent in the presence of a non-nucleophilic base to produce a compound of Formula IV; and 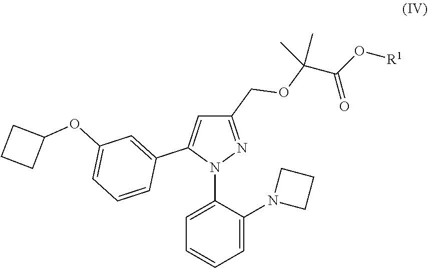
ii) hydrolyzing the compound of Formula IV to produce the compound of Formula I, or a salt thereof, wherein:
R¹ is C₁-C₄ alkyl; and
R² is halogen.

2. The process of claim 1, wherein R¹ is isopropyl and R² is bromine.

3. The process of claim 1 or 2, wherein the molar ratio of the compound of Formula III to the compound of Formula II is about 7.0 to about 10.0.

4. The process of any one of claims 1 to 3, wherein the non-nucleophilic base is chosen from N,N-diisopropylethylamine (DIPEA), 8-diazabicycloundec-7-ene (DBU), 1,5-diazabicyclo(4.3.0)non-5-ene (DBN), 2,6-di-tert-butylpyridine, tert-butyl-lithium, tert-butyl-phosphazene, lithium diisopropylamide (LDA), sodium bis(trimethylsilyl)amide (NaHMDS), potassium tert-butoxide, potassium bis(trimethylsilyl)amide (KHMDS), lithium tetramethylpiperidide (LiTMP), sodium hydride, potassium hydride, and sodium tert-butoxide.

5. The process of claim 4, wherein the non-nucleophilic base is potassium bis(trimethylsilyl)amide (KHMDS).

6. The process of claim 5, wherein the molar ratio of KHMDS to the compound of Formula II is about 4.0 to about 6.0.

7. The process of any one of claims 1 to 6, wherein the polar aprotic solvent is chosen from acetone, acetonitrile, dichloromethane, dimethyl sulfoxide (DMSO) dimethylformamide (DMF), ethyl acetate, hexamethylphosphoric triamide (HMPT), pyridine, tetrahydrofuran (THF), and mixtures thereof.

8. The process of claim 7, wherein the polar aprotic solvent is a mixture comprised of THF and DMF.

9. The process of any one of claims 1 to 8, wherein hydrolyzing the compound of Formula IV comprises reacting the compound of Formula IV with a nucleophilic base.

10. The process of claim 9, wherein the nucleophilic base is chosen from sodium hydroxide and sodium methoxide.

11. The process of claim 9 or 10, wherein the molar ratio of the nucleophilic base to the compound of Formula IV is about 18.0 to about 20.0.

12. The process of any one of claims 1 to 11, wherein the compound of Formula II is prepared by contacting a compound of Formula V: 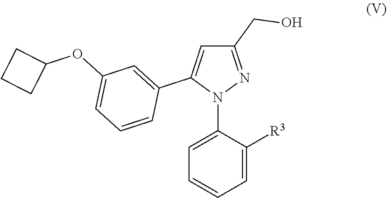
with azetidine in the presence of a palladium catalyst, wherein R³ is halogen.

13. The process of claim 12, wherein R³ is bromine.

14. The process of claim 12 or 13, wherein the molar ratio of azetidine to the compound of Formula V is about 2.0 to about 3.0.

15. The process of any one of claims 12 to 14, wherein the molar ratio of palladium catalyst to the compound of Formula V is about 0.05 to about 0.15.

16. The process of any one of claims 12 to 15, wherein the palladium catalyst is chosen from Pd(OAc)₂, palladium (II) pivalate, tetrakis(triphenylphosphine)palladium (0), bis(acetonitrile)palladium (II) dichloride, bis(triphenylphosphine)palladium (II) dichloride, [1,1′-bis(diphenylphosphino)ferrocene]palladium (II) dichloride, tris(dibenzylidenacetone)dipalladium (0), and palladium (II) chloride.

17. The process of claim 16, wherein the palladium catalyst is Pd(OAc)₂.

18. The process of any one of claims 12 to 17, wherein the contacting is conducted in the presence of a ligand.

19. The process of claim 18, wherein the ligand is chosen from trimethylphosphine, triphenylphosphine, tricyclohexylphosphine, tri(o-tolyl)phosphine, 2-(dicyclohexylphosphino)-2′,4′,6′-tri-i-propyl-1,1′-biphenyl (XPhos), 2-(dicyclohexylphosphino)-2′-(N,N-dimethylamino)biphenyl, 2-dicyclohexylphosphino-2′,6′-di-i-propoxy-1,1′-biphenyl, 2-dicyclohexylphosphino-2′,6′-dimethoxy-1,1′-biphenyl, butyldi-1-adamantylphosphine, 2-(di-t-butylphosphino)biphenyl, 2-(dicyclohexylphosphino)biphenyl, (R)-(−)-1-[(S)-2-(diphenylphosphino)ferrocenyl]ethyldicyclohexylphosphine, 1,2-bis(diphenylphosphino)benzene (dppbenzene), 2,2′-bis(diphenylphosphino)-1,1′-binaphthyl (BINAP), bis(2-diphenylphosphinophenyl)ether (DPEphos), 4,5-bis(diphenylphosphino)-9,9-dimethylxanthene (Xantphos), 1,4-bis(diphenylphosphino)butane (dppb), 1,2-bis(diphenylphosphino)ethane (dppe), 1,1′-bis(diphenylphosphino)ferrocene (dppf), 1,3-bis(diphenylphosphino)propane (dppp) and [(t-Bu₃)PH]BF₄.

20. The process of claim 19, wherein the ligand is Xantphos.

21. The process of any one of claims 18 to 20, wherein the molar ratio of ligand to the compound of Formula V is about 0.05 to about 0.15.

22. The process of any one of claims 12 to 21, wherein the compound of Formula V is prepared by contacting a compound of Formula VI 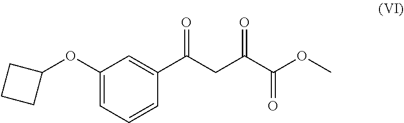 [SMILES: COC(=O)C(=O)CC(=O)c1cccc(OC2CCC2)c1]
with a compound of Formula VII: 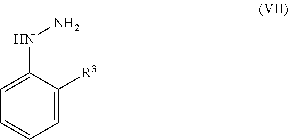
to form a compound of Formula VIII: 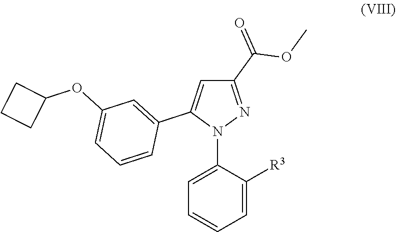
and subsequently contacting the compound of Formula VIII with a reducing agent to yield the compound of Formula V, wherein R³ is halogen.

23. The process of claim 22, wherein the reducing agent is chosen from lithium aluminum hydride, sodium bis(2-methoxyethoxy)aluminum hydride (Red-Al), nascent hydrogen, sodium amalgam, zinc amalgam, sodium borohydride, lithium borohydride, SmI₂, compounds containing the Fe²⁺ ion, compounds containing the Sn²⁺ ion, hydrazine, diisobutylaluminum hydride, and any combinations thereof.

24. The process of claim 23, wherein the reducing agent is sodium borohydride.

25. The process of any one of claims 22 to 24, wherein the compound of Formula VI is prepared by contacting 1-(3-hydroxyphenyl)ethan-1-one with a compound of Formula IX: 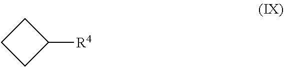
in the presence of a non-nucleophilic base in a polar aprotic solvent to form 1-(3-cyclobutoxyphenyl)ethan-1-one, which is subsequently contacted with dimethyl oxalate and a strong base to yield the compound of Formula VII, wherein R⁴ is halogen.

26. The process of claim 25, wherein the polar aprotic solvent is chosen from acetone, acetonitrile, dichloromethane, dimethyl sulfoxide (DMSO) dimethylformamide (DMF), ethyl acetate, hexamethylphosphoric triamide (HMPT), pyridine, tetrahydrofuran (THF), and mixtures thereof.

27. The process of claim 25 or 26, wherein the non-nucleophilic base is chosen from cesium carbonate, sodium carbonate, and potassium carbonate.

28. The process of claim 27, wherein the strong base is chosen from sodium hydroxide, sodium methoxide, sodium ethoxide, lithium diisopropylamide (LDA), sodium bis(trimethylsilyl)amide (NaHMDS), potassium tert-butoxide, potassium bis(trimethylsilyl)amide (KHMDS), lithium tetramethylpiperidide (LiTMP), sodium hydride, potassium hydride, and sodium tert-butoxide.

29. The process of any one of claims 25 to 29, wherein the molar ratio of non-nucleophilic base to 1-(3-hydroxyphenyl)ethan-1-one is about 1.0 to about 2.0.

30. The process of any one of the preceding claims, further comprising converting the compound of Formula I to a tris salt of formula

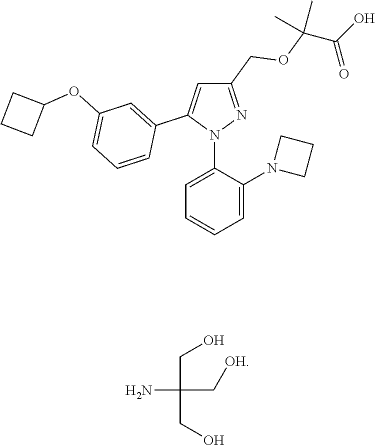 [SMILES: CC(C)(OCc1cc(-c2cccc(OC3CCC3)c2)n(-c2ccccc2N2CCC2)n1)C(=O)O ; NC(CO)(CO)CO]

31. A tris salt prepared by the process of claim 30.

32. A compound of formula 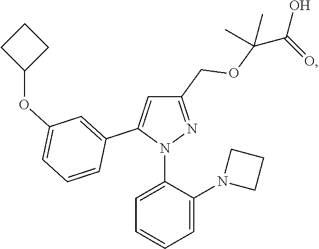 [SMILES: CC(C)(OCc1cc(-c2cccc(OC3CCC3)c2)n(-c2ccccc2N2CCC2)n1)C(=O)O]
prepared by the process of one any of claims 1-29, wherein the compound contains a detectable amount that is less than 3% by weight of one or more residual organic solvents.

33. A composition comprising at least 90% a compound of Formula I, or a salt thereof, and a detectable amount of one or more impurities chosen from a compound of Formula IV, a compound of Formula II, a compound of Formula V, a compound of Formula VIII, 1-(3-hydroxyphenyl)ethan-1-one, bromocyclobutane, cesium carbonate, hydrochloric acid, 2-bromophenylhydrazine, sodium borohydride, xantphos, sodium tert-butoxide, isopropyl 2-bromo-2-methylpropanoate, potassium bis(trimethylsilyl)amide, sodium hydroxide, sodium hydride, sodium tert-pentoxide, acetonitrile, dichloromethane, 4-dimethylaminopyridine, ethyl acetate, dimethylformamide, ethanol, water, isopropanol, isopropyl acetate, potassium bis(trimethylsilyl)amide, methanol, methyl tert-butyl ether, sodium borohydride, N-methyl-2-pyrrolidone, palladium diacetate, sodium triacetoxyborohydride, tetrahydrofuran, and one or more heavy metals.

34. The composition of claim 33, wherein the one or more heavy metals are chosen from platinum, palladium, iridium, rhodium, rhenium, ruthenium, cadmium, mercury, lead, arsenic, manganese, chromium, cobalt, nickel, copper, zinc, selenium, silver, antimony, thalium, nickel, vanadium, and zinc.

35. A method for treating a monocarboxylate transporter MCT4-mediated disorder in a subject in need thereof, comprising the step of administering to the subject therapeutically effective amounts of the compound of claim 32, or a salt thereof, the salt of claim 31, or the composition of claim 33 or 34.

36. The method as recited in claim 35, wherein the monocarboxylate transporter MCT4-mediated disorder is cardiac hypertrophy.

37. The method as recited in claim 35, wherein the monocarboxylate transporter MCT4-mediated disorder is heart failure.

38. The method as recited in claim 35, wherein the monocarboxylate transporter MCT4-mediated disorder is a cancer that expresses MCT4.

39. The method as recited in claim 35, wherein the monocarboxylate transporter MCT4-mediated disorder is rheumatoid arthritis.

40. A compound of formula 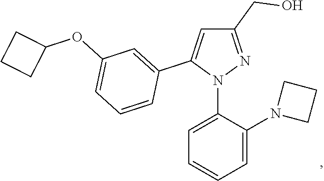 [SMILES: OCc1cc(-c2cccc(OC3CCC3)c2)n(-c2ccccc2N2CCC2)n1]
or a salt thereof.

41. A compound of formula 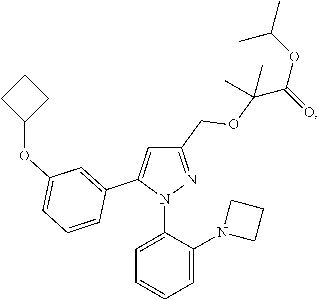 [SMILES: CC(C)OC(=O)C(C)(C)OCc1cc(-c2cccc(OC3CCC3)c2)n(-c2ccccc2N2CCC2)n1]
or a salt thereof.
